(19) United States
(12) Patent Application Publication
(10) Pub. No.: US 2026/0109679 A1
(43) Pub. Date: Apr. 23, 2026

(54) METHOD FOR PRODUCING HIGH-PURITY 2,5-FURANDIMETHANOL BY CATALYZING FURFURYL ALCOHOL USING APROTIC ACID CATALYST

(71) Applicant: INSTITUTE OF CHEMICAL INDUSTRY OF FOREST PRODUCTS, CAF, Nanjing (CN)

(72) Inventors: Kui WANG, Nanjing (CN); Shanyong WANG, Nanjing (CN); Jianchun JIANG, Nanjing (CN)

(73) Assignee: INSTITUTE OF CHEMICAL INDUSTRY OF FOREST PRODUCTS, CAF, Nanjing (CN)

(21) Appl. No.: 19/477,495

(22) PCT Filed: Sep. 6, 2024
(86) PCT No.: PCT/CN2024/117580

§ 371 Date: Oct. 22, 2025

(30) Foreign Application Priority Data
Jan. 10, 2024 (CN) 202410037023.3

(51) Int. Cl. C07D 307/42

(52) U.S. Cl. CPC C07D 307/42

(57) ABSTRACT

A method for producing high-purity 2,5-furandimethanol by catalyzing FAL using an aprotic acid catalyst is provided. The method includes: adding a modified zeolite catalyst, the FAL and a hydroxymethylation agent to an aprotic solvent, introducing nitrogen gas for reaction, filtering after the reaction to separate a solid phase and a liquid phase, rotary evaporating the liquid phase to recover the solvent, and then continuing to heat to collect a solid to obtain the high-purity 2,5-furandimethanol; where the modified zeolite catalyst is obtained by subjecting a β-zeolite to dealumination, followed by introducing a transition metal element as a Lewis acid active site into the zeolite via a solid-solid ion exchange method. As the aprotic acid catalyst, the modified zeolite can achieve high selectivity for the target product, featuring a simple synthesis process and separation-purification procedure, low raw material cost, and compliance with the development concept of green chemistry.

### CROSS REFERENCE TO THE RELATED APPLICATIONS

This application is the national phase entry of International Application No. PCT/CN2024/117580, filed on Sep. 6, 2024, which is based upon and claims priority to Chinese Patent Application No. 202410037023.3, filed on Jan. 10, 2024, the entire contents of which are incorporated herein by reference.

### TECHNICAL FIELD

This invention belongs to the field of furan-based biomass chemicals and materials, and specifically relates to a method for producing high-purity 2,5-furandimethanol by catalyzing furfuryl alcohol (FAL) using an aprotic acid catalyst.

### BACKGROUND

2,5-furandimethanol (2,5-bis(hydroxymethyl)furan, BHMF) is an aromatic furan diol primarily derived from 5-hydroxymethylfurfural (HMF). It is an important monomer for replacing petroleum-based diols in the synthesis of aromatic polyester materials and has a wide range of applications in the synthesis of ethers, polymers, resins, and adhesives.

Currently, the main factor limiting the application of BHMF is the high cost of HMF: the industrial production of HMF primarily relies on the dehydration of fructose using inorganic acids, while the cheaper glucose process is still under research. Furthermore, the reduction and derivatization of HMF require high selectivity for C═O and C═C bonds, which poses significant challenges for the catalyst and catalytic process. In contrast, the furfural industry has achieved economies of scale, with over 65% of its production capacity dedicated to FAL-related production applications. This means that directly producing BHMF through the hydroxymethylation of FAL is a highly cost-effective strategy.

The earliest known attempt to produce BHMF from FAL dates back to Laszlo-Hedwig et al. (Polymer Science U.S.S.R., 1983, 25:228-236). By mimicking the acidic polymerization process of phenolic resin, they used formaldehyde as the hydroxymethylation agent to convert FAL into BHMF in an acidic environment. However, compared to the hydroxymethylation of phenol, the highly reactive FAL undergoes severe self-polymerization in acidic environments, especially in aqueous environment, resulting in lower BHMF selectivity. Furthermore, side reactions triggered by the ring-opening of the furan structure in strong acidic environments are another significant factor contributing to the lower BHMF selectivity. To address these issues, researchers have conducted research: high-silica hydrophobic mordenite can replace inorganic acids, achieving relative high BHMF selectivity at low FAL concentrations; polyoxymethylene can replace formaldehyde solution to mitigate side reactions caused by the introduction of an aqueous phase due to formaldehyde; and protecting the active hydroxyl groups of FAL prior to hydroxymethylation can reduce related side reactions. Even so, several challenges remain: the high reaction barrier of the C—H bond on the aromatic ring, the self-polymerization of free formaldehyde after protonation, product selectivity, and subsequent separation challenges caused by byproducts such as humic acid.

Therefore, a method for preparing high-purity BHMF with high selectivity, simple separation, minimal side reactions, and a simple process is urgently needed.

### SUMMARY

### Technical Problem

In response to the above problems in the prior art, the technical problem to be solved by the present invention is to provide a method for producing high-purity 2,5-furandimethanol by catalyzing FAL using an aprotic acid catalyst. This method addresses the current problems of severe side reactions, poor selectivity, complex separation, and high cost in the production of 2,5-furandimethanol.

### Technical Solution

To address the above problems, the present invention adopts the following technical solution:

The method for producing the high-purity 2,5-furandimethanol by catalyzing the FAL using the aprotic acid catalyst, including adding a modified zeolite catalyst, the FAL, and a hydroxymethylation agent to an aprotic solvent, introducing nitrogen gas for reaction, filtering after the reaction to separate a solid phase and a liquid phase, rotary evaporating the liquid phase to recover the solvent, and then continuing to heat to collect a solid to obtain the high-purity 2,5-furandimethanol; where the modified zeolite catalyst is obtained by subjecting a β-zeolite to dealumination with concentrated nitric acid, followed by introducing a transition metal element into the β-zeolite via a solid-solid ion exchange method.

Furthermore, the transition metal element is one or more of manganese, cobalt, and tin.

Furthermore, the transition metal is the manganese.

Furthermore, the hydroxymethylation agent is one or a mixture of formaldehyde, trioxymethylene, and paraformaldehyde.

Furthermore, the aprotic solvent is one or a mixture of tetrahydrofuran, 1,4-dioxane, and methyl acetate.

Furthermore, a molar ratio of the FAL to the hydroxymethylation agent is 1:1-9.

Furthermore, an addition amount of the FAL is 1-10% of a volume of the aprotic solvent, and an addition amount of the modified zeolite catalyst is 1-20% of a total solvent mass.

Furthermore, a temperature of the reaction is 80-140° C., and a duration of the reaction is 1-12 h.

Furthermore, the specific steps are as follows:

1) dealuminating the β-zeolite in the concentrated nitric acid at boiling for 8 h, then washing with water until neutral and drying, introducing an exogenous ion, which is the transition metal element, by mechanical grinding, then calcining at a high temperature, and cooling to obtain the modified zeolite catalyst; where a molar ratio of SiO₂ to Al₂O₃ in the β-zeolite is 30:1;

2) adding the FAL and the hydroxymethylation agent to the aprotic solvent at a molar ratio of 1:1-9, together with the catalyst equivalent to 1-20% of a total solvent mass, and reacting for 1-12 h under conditions of the nitrogen gas as a protective gas and a temperature of 80-140° C.; where an addition amount of the FAL is 1-10% of a volume of the aprotic solvent; and

3) after the reaction, filtering to separate the solid and a liquid in a reaction system, washing the solid, which is the catalyst, with a washing solution and drying to recover, subjecting the liquid to rotary evaporation at 40-80° C. to separate the solvent, continue heating to 100° C. to remove trace amounts of FAL substrate, and collecting the solid to obtain the high-purity 2,5-furandimethanol.

Furthermore, in step 3), the washing solution is one or a mixture of methanol, ethanol, and acetone.

The schematic diagram of the conversion of the FAL into the high-purity 2,5-furandimethanol under the catalysis of the aprotic acid catalyst according to the present invention is shown in FIG. 1.

### Beneficial Effects

Beneficial effects: Compared with the prior art, the advantages of the present invention are:

(1) The catalyst used in the present invention is the aprotic acid catalyst, which is characterized by relying on Lewis acid active sites to complete the hydroxymethylation process, reducing protic acid-related side reactions.

(2) The catalytic pathway of the present invention is different from the hydroxymethylation process in which protic acid forms hydroxymethyl cations that then attack the FAL in a disordered manner. The aprotic acid catalyst of the present invention not only promotes the formation of the hydroxymethyl cations but also assists in anchoring them to the reaction site of the FAL, thereby achieving targeted completion of the hydroxymethylation reaction and reducing side reactions related to the electrophilic reagents.

(3) The low level of byproducts and high selectivity for the BHMF in the present invention allows the reaction solution and catalyst to be recovered by simple distillation, simplifying the product separation and reagent recovery processes.

(4) The synthesis process and separation-purification procedure of the present invention are simple, the catalyst can be recycled and reused, and the raw material cost is reduced, which is in line with the development concept of green chemistry.

### BRIEF DESCRIPTION OF THE DRAWINGS

FIG. 1 is a schematic diagram of the conversion of FAL into the high-purity 2,5-furandimethanol under the catalysis of the aprotic acid catalyst according to the present invention;

FIG. 2 is a gas chromatography-mass spectrometry (GC-MS) time spectrum of the reaction solution prepared according to the present invention;

FIG. 3 is a mass spectrogram corresponding to the BHMF product peak in FIG. 1 of the present invention;

FIG. 4 is a GC-MS time spectrum of the product prepared in Comparative Example 1 of the present invention.

### DETAILED DESCRIPTION OF THE EMBODIMENTS

The present invention is further described below with reference to specific examples.

The selectivity, yield, and purity of the BHMF prepared in the following examples were calculated using the following formula:

The selectivity of the BHMF (S_{BHMF}) was calculated using the following formula:

$${S}_{BHMF} = \frac{{m}_{3} {M}_{FAL}}{( {m}_{0} - {m}_{1} ) {M}_{BHMF}}$$

Where m₀ is the mass of the FAL substrate; m₁ is the mass of the remaining FAL substrate; m₃ is the mass of the BHMF, chromatographic grade; M_{FAL} is the molar mass of the FAL; and M_{BHMF} is the molar mass of the BHMF.

The BHMF yield, Y_{BHMF}, is calculated as follows:

$${Y}_{BHMF} = \frac{{m}_{2} {M}_{FAL}}{{m}_{0} {M}_{BHMF}}$$

Where m₂ is the mass of the absolute dry product; m₀ is the mass of the FAL substrate; M_{FAL} is the molar mass of the FAL; and M_{BHMF} is the molar mass of the BHMF.

The purity of the BHMF (P_{BHMF}) is calculated as follows:

$${P}_{BHMF} = \frac{{m}_{3}}{{m}_{2}}$$

Where m₃ is the mass of the BHMF determined by chromatography; m₂ is the mass of the absolute dry product.

The purity of the BHMF prepared in the following examples was determined by chromatographic purity, as follows:

0.1 g (absolute dry equivalent) of the product was placed in the beaker and dissolved in the appropriate amount of tetrahydrofuran. The mixture was then cooled to room temperature and diluted to 100 mL. The solution was filtered through the 0.22 μm organic syringe filter and used to determine the BHMF concentration. Analysis was performed using the gas chromatograph (Shimadzu, GC-2010 Plus) equipped with the highly polar column (DB-WAXetr). N-dodecane, chromatographic grade, was used as the internal reference. The test conditions were: the temperature range of 40-280° C., the heating rate of 5° C./min between 40° C. and 150° C. and the heating rate of 2° C./min between 150° C. and 280° C. The precise BHMF content in the product was obtained through the internal reference and the pre-established BHMF standard curve, and further calculation gives the chromatographic purity of the sample.

Formaldehyde (37 wt % aqueous solution), trioxymethylene (AR), paraformaldehyde (95%), tetrahydrofuran (AR), 1,4-dioxane (99.5%), and methyl acetate (99%) used in the following examples were purchased from Shanghai Macklin.

### Example 1

### 1. Preparation of the Modified Zeolite Catalyst

The β-zeolite (SiO₂ and Al₂O₃ in the molar ratio of 30:1) was placed in the concentrated nitric acid (68% by mass) and dealuminated at boiling for 8 h, followed by washing with the water until neutral and drying at 105° C. The cobalt nitrate in a molar aquivalent equal to the removed aluminum element was then added, and mechanical grinding was performed. The mixture was then treated in the tube furnace at 600° C. under the nitrogen gas for 4 h. The mixture was then placed in the muffle furnace and held at 600° C. for 4 h.

The product, after cooling, was the prepared modified zeolite catalyst.

### 2. Preparation of the BHMF

The FAL and the formaldehyde solution (based on the formaldehyde monomer molar equivalents) were added to the tetrahydrofuran at the molar ratio of 1:3, with the FAL accounting for 2% of the volume of the tetrahydrofuran. The modified zeolite catalyst equivalent to 5% of the total solvent mass was also added. The air in the apparatus was purged with the nitrogen gas, and the reaction was carried out by stirring at 100° C. for 4 h. After the reaction was completed, the solid and liquid phases were separated using the filtration device. The solid was continuously washed with the ethanol three times and then dried to recover the catalyst at 105° C. The liquid was then subjected to the rotary evaporator at 60° C. to recover the solvent. The temperature was then raised to 100° C. to remove the trace amounts of FAL substrate. The remaining crystals were collected to obtain the BHMF.

### Example 2

### Preparation of the BHMF

The FAL and the formaldehyde solution (based on the formaldehyde monomer molar equivalents) were added to the tetrahydrofuran at the molar ratio of 1:9, with the FAL accounting for 5% of the volume of the tetrahydrofuran. The modified zeolite catalyst (prepared according to the method of Example 1) equivalent to 10% of the total solvent mass was also added. The air in the apparatus was purged with the nitrogen gas, and the reaction was carried out by stirring at 80° C. for 8 h. After the reaction was completed, the solid and liquid phases were separated using the filtration device. The solid was continuously washed with the ethanol three times and then dried to recover the catalyst at 105° C. The liquid was then subjected to the rotary evaporator at 60° C. to recover the solvent. The temperature was then raised to 100° C. to remove the trace amounts of FAL substrate. The remaining crystals were collected to obtain the BHMF.

### Example 3

### Preparation of the BHMF

The FAL and the trioxymethylene (based on the formaldehyde monomer molar equivalents) were added to the tetrahydrofuran at the molar ratio of 1:9, with the FAL accounting for 5% of the volume of the tetrahydrofuran. The modified zeolite catalyst (prepared according to the method of Example 1) equivalent to 10% of the total solvent mass was also added. The air in the apparatus was purged with the nitrogen gas, and the reaction was carried out by stirring at 100° C. for 4 h. After the reaction was completed, the solid and liquid phases were separated using the filtration device. The solid was continuously washed with the acetone three times and then dried to recover the catalyst at 105° C. The liquid was then subjected to the rotary evaporator at 60° C. to recover the solvent. The temperature was then raised to 100° C. to remove the trace amounts of FAL substrate. The remaining crystals were collected to obtain the BHMF.

### Example 4

### Preparation of the BHMF

The FAL and the paraformaldehyde (based on the formaldehyde monomer molar equivalents) were added to the methyl acetate at the molar ratio of 1:3, with the FAL accounting for 5% of the volume of the methyl acetate. The modified zeolite catalyst (prepared according to the method of Example 1) equivalent to 10% of the total solvent mass was also added. The air in the apparatus was purged with the nitrogen gas, and the reaction was carried out by stirring at 100° C. for 8 h. After the reaction was completed, the solid and liquid phases were separated using the filtration device. The solid was continuously washed with the methanol three times and then dried to recover the catalyst at 105° C. The liquid was then subjected to the rotary evaporator at 60° C. to recover the solvent. The temperature was then raised to 100° C. to remove the trace amounts of FAL substrate. The remaining crystals were collected to obtain the BHMF.

### Example 5

### 1. Preparation of the Modified Zeolite Catalyst

The β-zeolite (SiO₂ and Al₂O₃ in the molar ratio of 30:1) was placed in the concentrated nitric acid (68% by mass) and dealuminated at boiling for 8 h, followed by washing with the water until neutral and drying at 105° C. The manganese nitrate in a molar aquivalent equal to the removed aluminum element was then added, and mechanical grinding was performed. The mixture was then placed in the muffle furnace and held at 600° C. for 4 h.

The product, after cooling, was the prepared modified zeolite catalyst.

### 2. Preparation of the BHMF

The FAL and the paraformaldehyde (based on the formaldehyde monomer molar equivalents) were added to the methyl acetate at the molar ratio of 1:9, with the FAL accounting for 5% of the volume of the methyl acetate. The modified zeolite catalyst equivalent to 10% of the total solvent mass was also added. The air in the apparatus was purged with the nitrogen gas, and the reaction was carried out by stirring at 120° C. for 8 h.

After the reaction was completed, the solid and liquid phases were separated using the filtration device. The solid was continuously washed with the ethanol three times and then dried to recover the catalyst at 105° C. The liquid was then subjected to the rotary evaporator at 60° C. to recover the solvent. The temperature was then raised to 100° C. to remove the trace amounts of FAL substrate. The remaining crystals were collected to obtain the BHMF.

### Example 6

### Preparation of the BHMF

The FAL and the paraformaldehyde (based on the formaldehyde monomer molar equivalents) were added to the methyl acetate at the molar ratio of 1:6, with the FAL accounting for 10% of the volume of the methyl acetate. The modified zeolite catalyst (prepared according to the method of Example 5) equivalent to 20% of the total solvent mass was added. The air in the apparatus was purged with the nitrogen gas, and the reaction was carried out by stirring at 120° C. for 8 h. After the reaction was completed, the solid and liquid phases were separated using the filtration device. The solid was continuously washed with the ethanol three times and then dried to recover the catalyst at 105° C. The liquid was then subjected to the rotary evaporator at 60° C. to recover the solvent. The temperature was then raised to 100° C. to remove the trace amounts of FAL substrate. The remaining crystals were collected to obtain the BHMF.

### Example 7

### Preparation of the BHMF

The FAL and the paraformaldehyde (based on the formaldehyde monomer molar equivalents) were added to the 1,4-dioxane at the molar ratio of 1:9, with the FAL accounting for 1% of the volume of the 1,4-dioxane. The modified zeolite catalyst (prepared according to the method of Example 5) equivalent to 5% of the total solvent mass was also added, and the reaction was carried out by stirring at 110° C. for 4 h. After the reaction was completed, the solid and liquid phases were separated using the filtration device. The solid was continuously washed with the ethanol three times and then dried to recover the catalyst at 105° C. The liquid was then subjected to the rotary evaporator at 60° C. to recover the solvent. The temperature was then raised to 100° C. to remove the trace amounts of FAL substrate. The remaining crystals were collected to obtain the BHMF.

### Example 8

### 1. Preparation of the Modified Zeolite Catalyst

The β-zeolite (SiO₂ and Al₂O₃ in the molar ratio of 30:1) was placed in the concentrated nitric acid (68% by mass) and dealuminated at boiling for 8 h, followed by washing with the water until neutral and drying at 105° C. The manganese nitrate in a molar aquivalent equal to the removed aluminum element was then added, and mechanical grinding was performed. The mixture was then treated in the tube furnace at 600° C. under the nitrogen gas for 4 h. The mixture was then placed in the muffle furnace and held at 600° C. for 4 h. The product, after cooling, was the prepared modified zeolite catalyst.

### 2. Preparation of the BHMF

The FAL and the paraformaldehyde (based on the formaldehyde monomer molar equivalents) were added to the 1,4-dioxane at the molar ratio of 1:9, with the FAL accounting for 1% of the volume of the 1,4-dioxane. The modified zeolite catalyst equivalent to 5% of the total solvent mass was also added. The air in the apparatus was purged with the nitrogen gas, and the reaction was carried out by stirring at 110° C. for 12 h. After the reaction was completed, the solid and liquid phases were separated using the filtration device. The solid was continuously washed with the ethanol three times and then dried to recover the catalyst at 105° C. The liquid was then subjected to the rotary evaporator at 80° C. to recover the solvent. The temperature was then raised to 100° C. to remove the trace amounts of FAL substrate. The remaining crystals were collected to obtain the BHMF.

### Example 9

### 1. Preparation of the Modified Zeolite Catalyst

The β-zeolite (SiO₂ and Al₂O₃ in the molar ratio of 30:1) was placed in the concentrated nitric acid (68% by mass) and dealuminated at boiling for 8 h, followed by washing with the water until neutral and drying at 105° C. The tin nitrate in a molar aquivalent equal to the removed aluminum element was then added, and mechanical grinding was performed. The mixture was then treated in the tube furnace at 600° C. under the nitrogen gas for 4 h. The mixture was then placed in the muffle furnace and held at 600° C. for 4 h. The product, after cooling, was the prepared modified zeolite catalyst.

### 2. Preparation of the BHMF

The FAL and the paraformaldehyde (based on the formaldehyde monomer molar equivalents) were added to the 1,4-dioxane at the molar ratio of 1:9, with the FAL accounting for 1% of the volume of the 1,4-dioxane. The modified zeolite catalyst equivalent to 5% of the total solvent mass was also added. The air in the apparatus was purged with the nitrogen gas, and the reaction was carried out by stirring at 80° C. for 12 h. After the reaction was completed, the solid and liquid phases were separated using the filtration device. The solid was continuously washed with the acetone three times and then dried to recover the catalyst at 105° C. The liquid was then subjected to the rotary evaporator at 60° C. to recover the solvent. The temperature was then raised to 100° C. to remove the trace amounts of FAL substrate. The remaining crystals were collected to obtain the BHMF.

### Comparative Example 1

The FAL and the paraformaldehyde (based on the formaldehyde monomer molar equivalents) were added to the 1,4-dioxane at the molar ratio of 1:9, with the FAL accounting for 5% of the volume of the 1,4-dioxane. The acetic acid equivalent to 5% of the total solvent volume was also added as the catalyst. The air in the apparatus was purged with the nitrogen gas, and the reaction was carried out by stirring at 110° C. for 6 h. After the reaction was complete, the solvent and the acetic acid were recovered using the rotary evaporator at 80° C. The temperature was then raised to 100° C. to remove the trace amounts of FAL substrate. The remaining crystals were collected to obtain the BHMF.

### Comparative Example 2

The FAL and the paraformaldehyde (based on the formaldehyde monomer molar equivalents) were added to the 1,4-dioxane at the molar ratio of 1:9, with the FAL accounting for 1% of the volume of the 1,4-dioxane. The unmodified β-zeolite catalyst (SiO₂ and Al₂O₃ in the molar ratio of 30:1) equivalent to 20% of the total solvent mass was also added. The air in the apparatus was purged with the nitrogen gas, and the reaction was carried out by stirring at 110° C. for 6 h. After the reaction was completed, the filtration was performed. The solid was continuously washed with the acetone three times and then dried to recover the catalyst at 105° C. The liquid was then subjected to the rotary evaporator at 60° C. to recover the solvent. The temperature was then raised to 100° C. to remove the trace amounts of FAL substrate. The remaining crystals were collected to obtain the BHMF.

### Comparative Example 3

The FAL and the paraformaldehyde (based on the formaldehyde monomer molar equivalents) were added to the 1,4-dioxane at the molar ratio of 1:9, with the FAL accounting for 1% of the volume of the 1,4-dioxane. The zeolite catalyst (H-ZSM-5, SiO₂ and Al₂O₃ in the molar ratio of 18:1) equivalent to 20% of the total solvent mass was also added. The air in the apparatus was purged with the nitrogen gas, and the reaction was carried out by stirring at 110° C. for 6 h. After the reaction was completed, the filtration was performed. The solid was continuously washed with the acetone three times and then dried to recover the catalyst at 105° C. The liquid was then subjected to the rotary evaporator at 60° C. to recover the solvent. The temperature was then raised to 100° C. to remove the trace amounts of FAL substrate. The remaining crystals were collected to obtain the BHMF.

### Comparative Example 4

The FAL and the paraformaldehyde (based on the formaldehyde monomer molar equivalents) were added to the 1,4-dioxane at the molar ratio of 1:9, with the FAL accounting for 100 of the volume of 1,4-dioxane. The zeolite catalyst (H-SAPO-11, with the molar ratio of SiO₂, Al₂O₃, and P₂O₅ of 0.63:1:0.82) equivalent to 200% of the total solvent mass was also added. The air in the apparatus was purged with nitrogen gas, and the reaction was carried out by stirring at 110° C. for 6 h. After the reaction was completed, the filtration was performed. The solid was continuously washed with the acetone three times and then dried to recover the catalyst at 105° C. The liquid was then subjected to the rotary evaporator at 60° C. to recover the solvent. The temperature was then raised to 100° C. to remove the trace amounts of FAL substrate. The remaining crystals were collected to obtain the B6.4124.

The product yield and the product conversion of the BHMF prepared in Examples 1-9 and Comparative Examples 1-4 of the present invention are shown in Table 1 below.

Table 1 the product yield and the product conversion of the BHMF prepared in Examples 1-9 and Comparative Examples 1-4

| | Conversion | Yield of | Purity of |
|---|---|---|---|
| | of the | the BHMF | the BHMF |
| | FAL (mol %) | (mol %) | (wt %) |
| Example 1 | 67.32 | 41.24 | 47.62 |
| Example 2 | 74.28 | 46.21 | 83.22 |
| Example 3 | 63.24 | 10.15 | 40.02 |
| Example 4 | 80.97 | 78.69 | 97.62 |
| Example 5 | 89.74 | 87.12 | 96.44 |
| Example 6 | 98.16 | 92.15 | 98.09 |
| Example 7 | 100 | 90.09 | 98.17 |
| Example 8 | 97.82 | 94.01 | 98.89 |
| Example 9 | 77.44 | 74.01 | 96.97 |
| Comparative | 100 | 76.97 | 80.04 |
| Example 1 | | | |
| Comparative | 100 | 74.68 | 77.64 |
| Example 2 | | | |
| Comparative | 100 | 70.91 | 74.14 |
| Example 3 | | | |
| Comparative | 100 | 71.58 | 69.81 |
| Example 4 | | | |

Table 1 shows the product yield and the product conversion of the BHMF prepared in Examples 1-9 and Comparative Examples 1-4. As can be seen from the table, the temperature and the duration of the catalytic reaction influence the progress and selectivity of the hydroxymethylation reaction. Generally, increasing the substrate concentration is detrimental to improving the selectivity of the reaction, while increasing the catalyst dosage can accelerate the progress of the reaction. Compared to the protic acid catalysts, such as those in Comparative Example 1, the BHMF produced by the aprotic acid catalyst has higher purity and allows for easier recovery of the catalyst, thus ensuring the purity of the target product and efficient separation and recovery. Furthermore, the silicon-aluminum ratio affects the amount of ion exchange. The high silicon-aluminum ratio results in fewer active sites, but the catalyst is typically used in excess, resulting in significant differences in catalytic performance. Among the different single metal-modified zeolite catalysts, the manganese-modified zeolite catalyst performed the best. In Comparative Examples 1-4, while the FAL conversion was high, the yield and the purity of the BHMF were low, failing to meet application requirements.

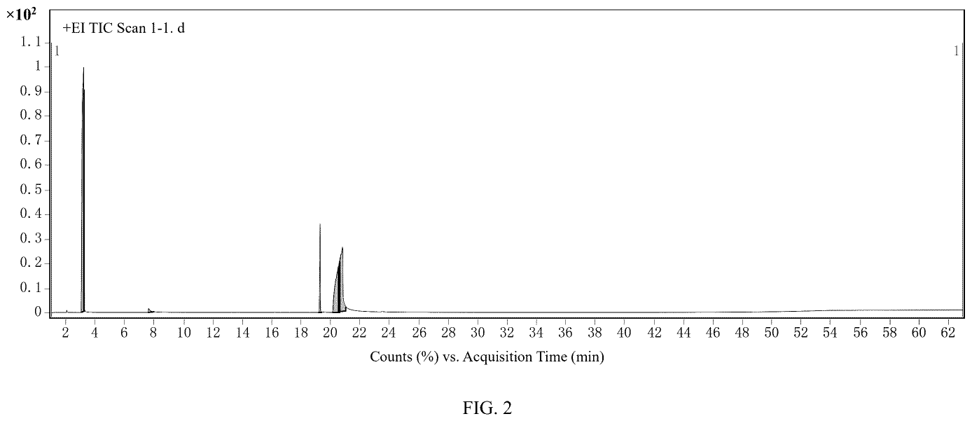

FIG. 2 is the GC-MS time spectrum of the reaction solution prepared in the present invention. As can be seen, the peaks at 3.185 min, 19.289 min, and 20.805 min represent the solvent peak, the internal reference peak, and the BHMF product peak, respectively. No obvious other byproduct peaks were observed.

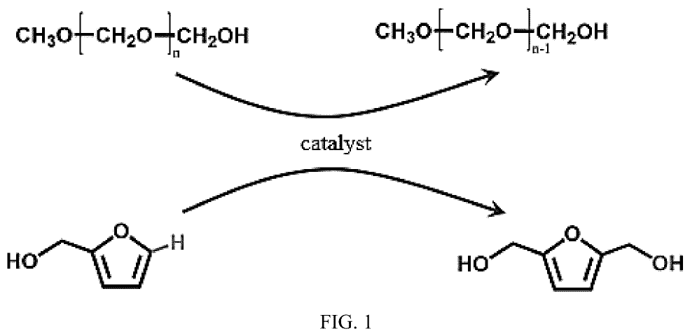

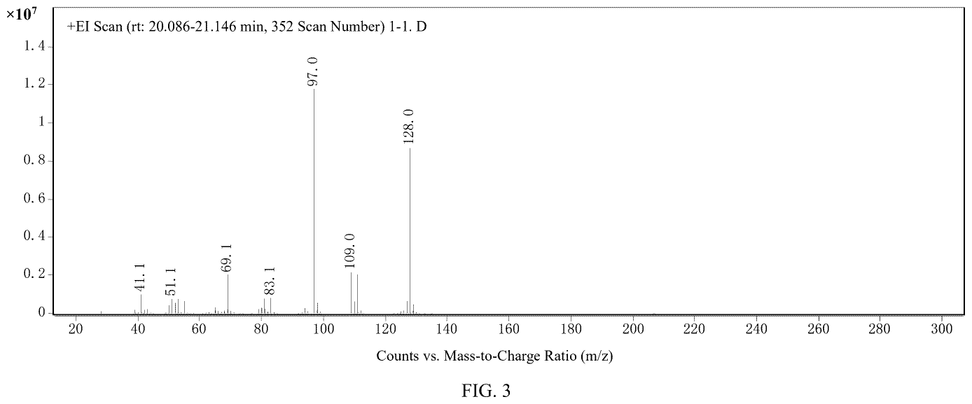

FIG. 3 is the mass spectrogram corresponding to the BHMF product peak in FIG. 1 of the present invention. As can be seen, the product at this position was identified as the BHMF based on comparison with the standard spectral library.

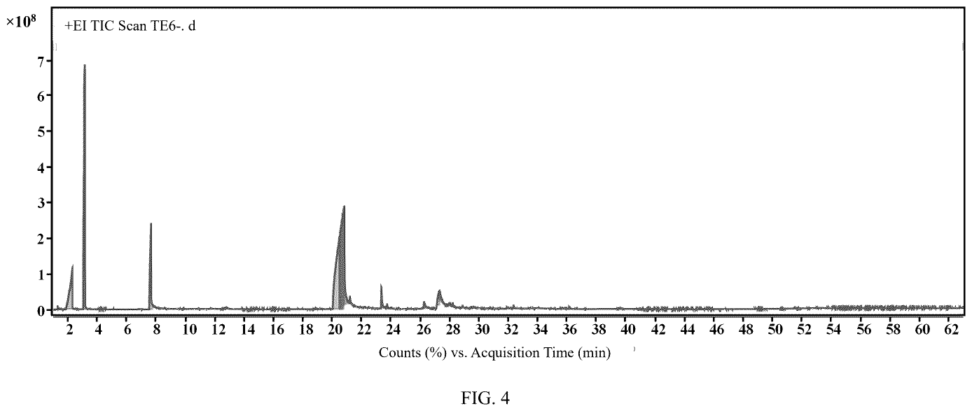

FIG. 4 is the GC-MS time spectrum of the product prepared in Comparative Example 1 of the present invention. As can be seen, excluding the solvent, substrate, and product peaks, the time spectrum under these experimental conditions displays numerous miscellaneous peaks within the time range. These miscellaneous peaks typically correspond to byproducts such as the acid polymerization and the hydrolysis of the FAL substrate, which can complicate the subsequent separation and purification.

1. A method for producing high-purity 2,5-furandimethanol by catalyzing furfuryl alcohol (FAL) using an aprotic acid catalyst, comprising adding a modified zeolite catalyst, the FAL, and paraformaldehyde to an aprotic solvent, introducing nitrogen gas for reaction, filtering after the reaction to separate a solid phase and a liquid phase, performing a rotary evaporation on the liquid phase to recover the solvent, and then continuing to heat to collect a solid as the high-purity 2,5-furandimethanol; wherein the modified zeolite catalyst is obtained by subjecting a β-zeolite to dealumination with concentrated nitric acid, followed by introducing a transition metal element into the β-zeolite via a solid-solid ion exchange method; the transition metal element is one or more of manganese, cobalt, and tin; and a temperature of the reaction is 100-140° C., and a duration of the reaction is 8-12 h.

2. The method for producing the high-purity 2,5-furandimethanol by catalyzing the FAL using the aprotic acid catalyst according to claim 1, wherein the transition metal is the manganese.

3. The method for producing the high-purity 2,5-furandimethanol by catalyzing the FAL using the aprotic acid catalyst according to claim 1, wherein the aprotic solvent is one or a mixture of tetrahydrofuran, 1,4-dioxane, and methyl acetate.

4. The method for producing the high-purity 2,5-furandimethanol by catalyzing the FAL using the aprotic acid catalyst according to claim 1, wherein a molar ratio of the FAL to the paraformaldehyde is 1:1-9.

5. The method for producing the high-purity 2,5-furandimethanol by catalyzing the FAL using the aprotic acid catalyst according to claim 1, wherein an addition amount of the FAL is 1-10% of a volume of the aprotic solvent, and an addition amount of the modified zeolite catalyst is 1-20% of a total solvent mass.

6. The method for producing the high-purity 2,5-furandimethanol by catalyzing the FAL using the aprotic acid catalyst according to claim 1, wherein the specific steps are as follows:
1) dealuminating the β-zeolite in the concentrated nitric acid at boiling for 8 h, then washing with water until neutral and drying, introducing an exogenous ion, which is the transition metal element, by mechanical grinding, then calcining at a high temperature, and cooling to obtain the modified zeolite catalyst; wherein a molar ratio of SiO₂ to Al₂O₃ in the β-zeolite is 30:1;
2) adding the FAL and the paraformaldehyde to the aprotic solvent at a molar ratio of 1:1-9, together with the catalyst equivalent to 1-20% of a total solvent mass, and reacting for 8-12 h under conditions of the nitrogen gas as a protective gas and the temperature of 100-140° C.; wherein an addition amount of the FAL is 1-10% of a volume of the aprotic solvent; and
3) after the reaction, filtering to separate the solid and the liquid in a reaction system, washing the solid, which is the catalyst, with a washing solution and drying to recover, subjecting the liquid to the rotary evaporation at 40-80° C. to separate the solvent, continuously heating to 100° C. to remove trace amounts of FAL substrate, and collecting the solid as the high-purity 2,5-furandimethanol.

7. The method for producing the high-purity 2,5-furandimethanol by catalyzing the FAL using the aprotic acid catalyst according to claim 6, wherein in the step 3), the washing solution is one or a mixture of methanol, ethanol, and acetone.
